(19) United States
(12) Patent Application Publication
(10) Pub. No.: US 2007/0233068 A1
(43) Pub. Date: Oct. 4, 2007

(54) Intervertebral prosthetic assembly for spinal stabilization and method of implanting same

(75) Inventors: Aurelien Bruneau, Memphis, TN (US); Thomas Carls, Memphis, TN (US); Eric C. Lange, Collierville, TN (US); Fred J. Molz IV, Birmingham, AL (US); Matthew M. Morrison, Cordova, TN (US); Jonathan Dewey, Memphis, TN (US); Kent M. Anderson, Memphis, TN (US)

Correspondence Address: HAYNES AND BOONE, LLP, 901 MAIN STREET, SUITE 3100, DALLAS, TX, 75202, US

(73) Assignee: SDGI Holdings, Inc., Wilmington, DE (US)

(21) Appl. No.: 11/359,070

(22) Filed: Feb. 22, 2006

(51) Int. Cl. A61F 2/30

(52) U.S. Cl. 606/61

(57) ABSTRACT

A prosthetic assembly and method of implanting same, according to which a least one rod is secured to the spinal column. A spacer engages the spinous process of a vertebrae of the spinal column. The rod is connected to the spacer via an adapter.

### BACKGROUND

The present invention relates to an intervertebral prosthetic assembly for stabilizing the human spine, and a method of implanting same.

Intervertebral discs that extend between adjacent vertebrae in vertebral columns of the human body provide critical support between the adjacent vertebrae while permitting multiple degrees of motion. These discs can rupture, degenerate, and/or protrude by injury, degradation, disease, or the like to such a degree that the intervertebral space between adjacent vertebrae collapses as the disc loses at least a part of its support function, which can cause impingement of the nerve roots and severe pain.

In some situations it is often necessary to perform a laminectomy to remove the laminae and the spinous process from at least one vertebrae to remove a intervertebral disc and/or to decompress a nerve root. Typically, in these procedures, two vertebral segments are fused together to stop any motion between the segments and thus relieve the pain.

Intervertebral prosthetic devices have been designed that can be implanted between the adjacent vertebrae, both anterior and posterior of the column. Many of these devices are supported between the spinous processes of the adjacent vertebrae to prevent the collapse of the intervertebral space between the adjacent vertebrae and provide motion stabilization of the spine. However, in the above situation involving removal of a spinous process from one of the vertebrae, it would be impossible to implant an intervertebral prosthetic device of the above type since the device requires support from the respective spinous processes of both adjacent vertebrae.

### SUMMARY

According to an embodiment of the invention, an intervertebral prosthetic assembly is provided that is implantable between two adjacent vertebrae to provide motion stabilization, despite the fact that at least one of vertebrae is void of a spinous process.

Various embodiments of the invention may possess one or more of the above features and advantages, or provide one or more solutions to the above problems existing in the prior art.

### BRIEF DESCRIPTION OF THE DRAWINGS

FIG. 1 is a side elevational view of an adult human vertebral column.

FIG. 2 is a posterior elevational view of the column of FIG. 1.

FIG. 3 is an enlarged, front elevational view of one of the vertebrae of the column of FIGS. 1 and 2.

FIG. 4 is an isometric view of a portion of the column of FIGS. 1 and 2, including the lower three vertebrae of the column, and depicting an intervertebral prosthetic assembly according to an embodiment of the invention implanted between two adjacent vertebrae.

FIG. 5 is an enlarged view of a portion of the column and the assembly shown in FIG. 4.

FIG. 6 is an enlarged isometric view of a component of the assembly of FIGS. 4 and 5.

FIGS. 7-9 are enlarged, isometric views of three alternate embodiments of the component of FIG. 6.

FIG. 10 is a view similar to that of FIG. 5, but depicting an alternate embodiment of the invention.

### DETAILED DESCRIPTION

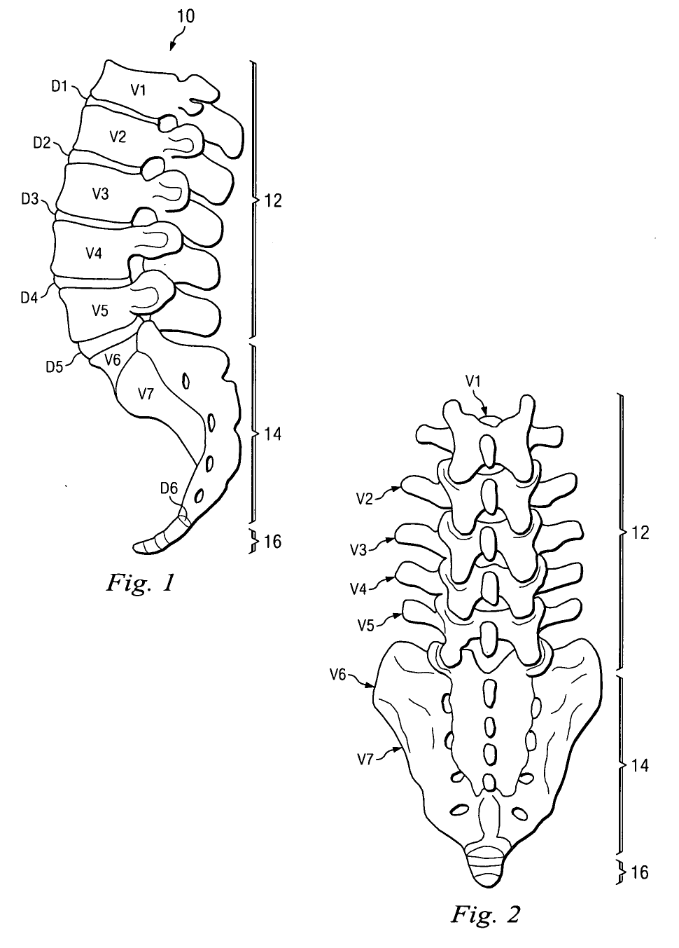

With reference to FIGS. 1 and 2, the reference numeral 10 refers, in general, to a human vertebral column 10. The lower portion of the vertebral column 10 is shown and includes the lumbar region 12, the sacrum 14, and the coccyx 16. The flexible, soft portion of the vertebral column 10, which includes the thoracic region and the cervical region, is not shown.

The lumbar region 12 of the vertebral column 10 includes five vertebrae V1, V2, V3, V4 and V5 separated by intervertebral discs D1, D2, D3, and D4, with the disc D1 extending between the vertebrae V1 and V2, the disc D2 extending between the vertebrae V2 and V3, the disc D3 extending between the vertebrae V3 and V4, and the disc D4 extending between the vertebrae V4 and V5.

The vertebrae V6 includes five fused vertebrae, one of which is a superior vertebrae V6 separated from the vertebrae V5 by a disc D5. The other four fused vertebrae of the sacrum 14 are referred to collectively as V7. A disc D6 separates the vertebrae V6 from the coccyx 16 that includes four fused vertebrae (not referenced).

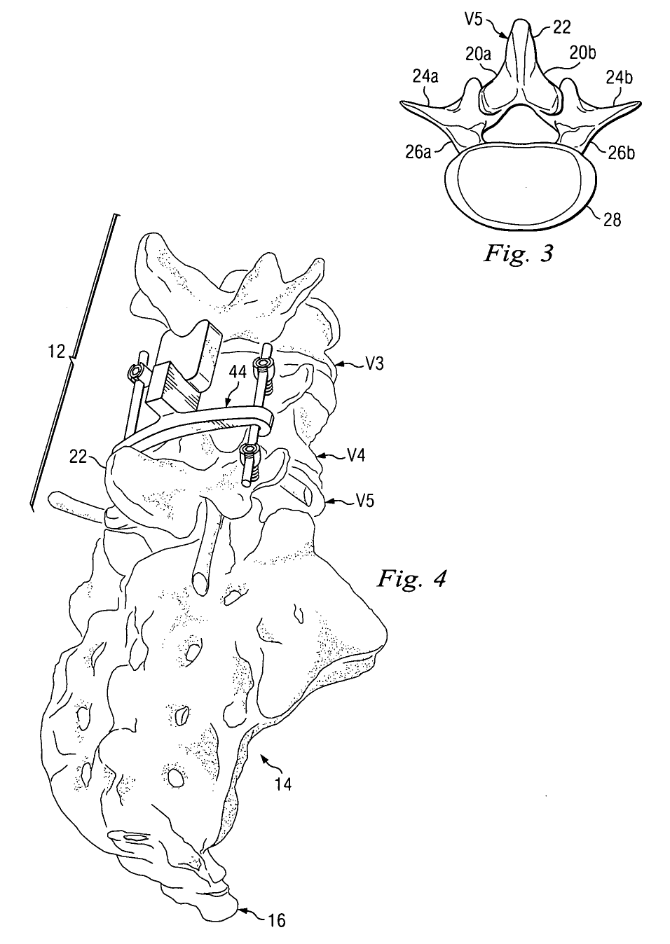

With reference to FIG. 3, the vertebrae V5 includes two laminae 20a and 20b extending to either side (as viewed in FIG. 2) of a spinous process 22 that projects posteriorly from the juncture of the two laminae. Two transverse processes 24a and 24b extend laterally from the laminae 20a and 20b, respectively, and two pedicles 26a and 26b extend anteriorly from the processes 24a and 24b to a vertebral body 28. Since the other vertebrae V1-V3 are similar to the vertebrae V5, they will not be described in detail. Also, V4 is similar to V5 with the exception that the spinous process 22 of V4 has been removed for one or both of the reasons set forth above.

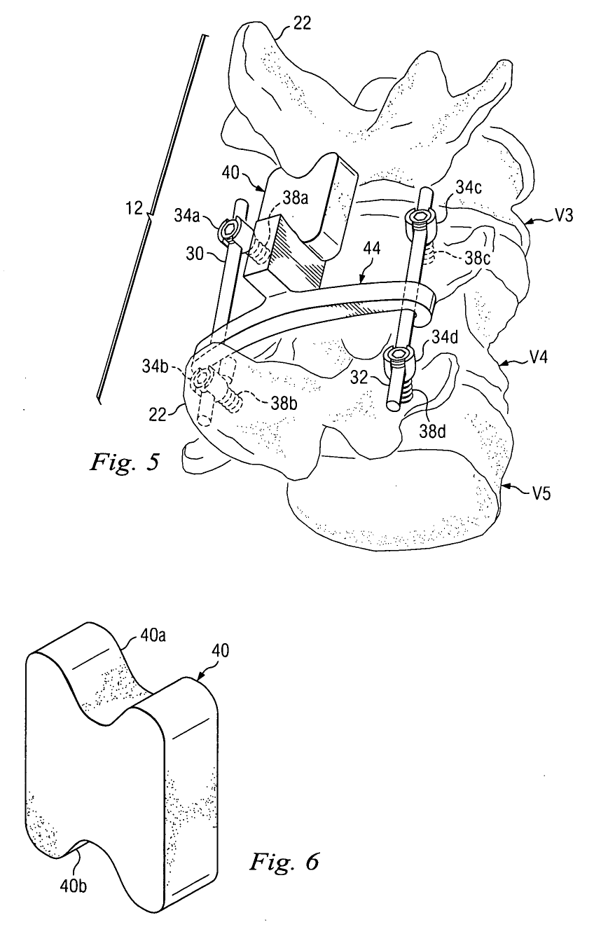

Referring to FIGS. 4 and 5, it will be assumed that, for one or more of the reasons set forth above, the vertebrae V4 and V5 are not being adequately supported by the disc D4, the spinous process 22 of V4 has been removed, and that it is desired to provide supplemental support and motion stabilization of these vertebrae.

To this end, two spaced, parallel, flexible rods 30 and 32 are provided that generally span the axial length between the processes 22 of the vertebrae V4 and V5. Two axially-spaced screw retainers 34a and 34b are connected to the rod 30 and two axially-spaced screw retainers 34c and 34d are connected to the rod 32. The screw retainers 34a, 34b, 34c, and 34d retain pedicle screws 38a, 38b, 38c, and 38d respectively, each of which extends through, and is supported by, its corresponding retainer.

The screws 38a and 38c extend into the pedicles of the vertebrae V4, and the screws 38b and 38d extend into the pedicles of the vertebrae V5. It is understood that the rods 30 and 32, the retainers 34a-34d and the screws 38a-38d are installed in connection with the procedure to be described, or that they could have been previously installed in connection with another procedure.

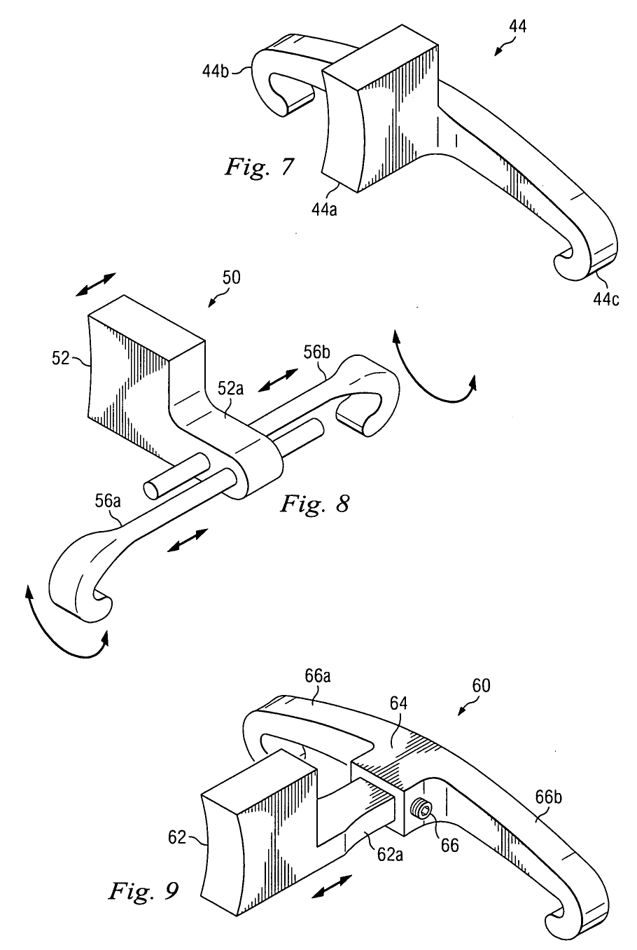

As shown in FIGS. 5 and 6, a spacer 40 is provided that is fabricated from a relatively flexible, soft material, and is substantially rectangular in shape with the exception that two curved notches, or saddles, 40a and 40b are formed at its respective end portions. The notch 40a extends around the spinous process 22 of the vertebrae V3, and, since the spinous process of the vertebrae V4 has been removed, an adapter 44, shown in detail in FIG. 7, is provided for supporting the spacer 40.

The adapter 44 comprises a rectangularly-shaped body member 44a that is sized so as to extend in the notch 40a of the spacer 40. Two arms 44b and 44c extend from the body member and can be formed integrally with, or attached to, the body member 44a. The respective distal end portions of the arms 44b and 44c curve downwardly from the body member as viewed in FIG. 7, and their respective distal end portions are curved inwardly so as to fit over the rods 30 and 32 (FIG. 5). Preferably, the adapter 44 is fabricated from a relatively stiff material, such as hard rubber or plastic.

The adapter 44 can be moved axially up or down the vertebral column 10 as necessary by moving the arms 44b and 44c along the rods 30 and 32, to insure that the spacer 40 fits between the spinous process 22 of the vertebrae V3 and the body member 44a of the adapter.

In its implanted position shown in FIG. 5, the assembly consisting of the rods 30 and 32, the spacer 40 and the adapter 44 stabilizes the vertebrae V3 and V4. Also, the relatively flexible, soft spacer 40 readily conforms to the processes 22 of the vertebrae V3 and provides excellent deformability resulting in an improved fit. The adapter 44 adds stiffness, compressive strength and durability, and the arms 44b and 44c restrain the adapter 44 from lateral movement.

An alternate embodiment of an adapter is shown, in general, by the reference numeral 50 in FIG. 8. The adapter 50 comprises a rectangularly-shaped body member 52 having a tab 52a extending from one end thereof. Two through-openings are provided in the tab 52a that receive two arms 56a and 56b, respectively. The arms 56a and 56b thus extend laterally from the body member 52, with their respective distal end portions being curved inwardly. The arms 56a and 56b extend in the openings in the tab 52a in a friction fit, and therefore can be adjusted laterally by moving them axially in the openings. Also, the angular position of the arms 56a and 56b relative to the body member 52 can be adjusted by rotating the arms in the openings in the tab 52a. If necessary, set screws (not shown), or the like, could be provided through additional openings in the tab 52a to lock the arms 56a and 56b in a desired axial and angular position. Preferably, the adapter 50 is fabricated from a relatively stiff material, such as hard rubber or plastic.

When the adapter 50 is used in place of the adapter 44 in the implanted position shown in FIG. 5, the spinous process 22 of the vertebrae V3 extends in the notch 40b of the spacer 40, and the body member 52 extends in the notch 40a. The effective lengths of the arms 56a and 56b can be adjusted so that their respective curved distal end portions extend over the rods 30 and 32, respectively.

The arms 56a and 56b prevent lateral movement of the adapter 50 yet permit the adapter 44 to be moved axially up or down the vertebral column 10 by moving the arms along the rods 30 and 32. Thus, the axial position of the adapter 50 can be adjusted as necessary to insure that the spacer 40 fits between the spinous process 22 of the vertebrae V3 and the body member 52 of the adapter.

The assembly consisting of the rods 30 and 32, the spacer 40, and the adapter 50 thus stabilizes the vertebrae V3 and V4. Also, the relatively flexible, soft spacer 40 readily conforms to the process 22 of the vertebrae V3 and provides excellent deformability resulting in an improved fit, while the adapter 50 adds stiffness, compressive strength and durability, and the arms 56a and 56b also restrain the adapter 44 from lateral movement.

Another alternate embodiment of an adapter is shown, in general, by the reference numeral 60 in FIG. 9. The adapter 60 comprises a rectangularly-shaped body member 62 having a stem 62a projecting therefrom and extending in an axial opening in a bracket 64. The lengths of the stem 60a and the latter opening are such that the amount of stem 60a that extends in the opening can be varied to vary the relative axial positions between the body member 62 and the bracket 64. A set screw 66 extends through a lateral opening in the bracket 64 and engages the stem 60a to lock the stem, and therefore the body member 62 to the bracket 64.

Two arms 66a and 66b extend laterally from the bracket 64 and preferably are formed integrally with the bracket. The arms 66a and 66b curve downwardly as viewed in FIG. 9, with their respective distal end portions being curved inwardly so as to fit over the rods 30 and 32 (FIG. 5). The arms 66a and 66b can be formed integrally with, or attached to, the bracket 64. Preferably, the adapter 60 is fabricated from a relatively stiff material, such as hard rubber or plastic.

When the adapter 60 is used in place of the adapter 44 in the implanted position shown in FIG. 5, the spinous process 22 of the vertebrae V3 extends in the notch 40b of the spacer 40, the body member 62 extends in the notch 40a, and the curved distal end portions of the arms 66a and 66b extend around the rods 30 and 32, respectively.

The arms 66a and 66b prevent lateral movement of the adapter 60 yet permit the adapter to be moved axially up or down the vertebral column 10 by moving the arms along the rods 30 and 32. Thus, the axial position of the adapter 60 can be adjusted as necessary to insure that the spacer 40 fits between the spinous process 22 of the vertebrae V3 and the body member 62 of the adapter.

The assembly consisting of the rods 30 and 32, the spacer 40 and the adapter 60 stabilizes the vertebrae V3 and V4. Also, the relatively flexible, soft spacer 40 readily conforms to the processes 22 of the vertebrae V3 and provides excellent deformability resulting in an improved fit, while the adapter 60 adds stiffness, compressive strength and durability, and the arms 66a and 66b restrain the adapter 44 from lateral movement.

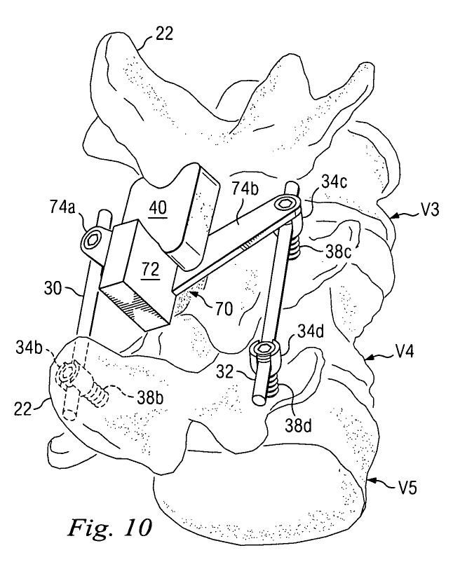

The embodiment of FIG. 10 is similar to that of FIG. 5 and includes identical components that are given the same reference numerals. According to the embodiment of FIG. 10, an adapter 70 is provided that consists of a rectangularly-shaped body member 72 that receives two arms 74a and 74b, respectively. The proximal ends of the arms 74a and 74b are connected to, or are formed integrally with, the body member 72, and the arms extend from the body member to the retainers 34a and 34c, respectively and thus extend at an acute angle with respect to the longitudinal axis of the column 12 (FIG. 2). The respective distal end portions of the arms 74a and 74b are connected to the screws 38a and 38c, respectively, and/or the retainers 34a and 34c, respectively in any conventional manner.

Assuming the spinous process 22 has been removed from the vertebrae V4 for one or more reasons set forth above, the adapter 70 is by positioning the spinous process 22 of the vertebrae V3 in the notch 40a of the spacer 40, and the body member 72 in the notch 42b. The distal end portions of the arms 76a and 76b are fastened to the retainers 34a and 34c, respectively to restrain the adapter 70 from lateral movement.

The assembly consisting of the rods 30 and 32, the spacer 40, and the adapter 70 thus stabilizes the vertebrae V3 and V4. Also, the relatively flexible, soft spacer 40 readily conforms to the process 22 of the vertebrae V3 and provides excellent deformability resulting in an improved fit, the adapter 70 adds stiffness, compressive strength and durability, and the arms 76a and 76b restrain the adapter 44 from lateral movement.

### Variations

It is understood that variations may be made in the foregoing without departing from the invention and examples of some variations are as follows:
The arms in each of the previous embodiments can be rigidly connected to their corresponding rods by set screws, or other connection devices.
The components disclosed above can be fabricated from materials other than those described above and may include a combination of soft and rigid materials.
The spacer in each of the above embodiments may be formed integrally with its corresponding adapter.
Any conventional substance that promotes bone growth, such as HA coating, BMP, or the like, can be incorporated in the above embodiments.
The surfaces of the spacer 40 defining the notches 40a and 42b can be treated, such as by providing teeth, ridges, knurling, etc., to better grip the spinous processes and the adapters.
The spacer 40 can be fabricated of a permanently deformable material thus providing a clamping action against the spinous processes 22.
One or more of the components disclosed above may have through-holes formed therein to improve integration of the bone growth.
The components of one or more of the above embodiments may vary in shape, size, composition, and physical properties.
Through-openings can be provided through one or more components of each of the above embodiments to receive tethers for attaching the devices to a vertebrae or to a spinous process.
The assemblies of each of the above embodiments can be placed between two vertebrae in the vertebral column 10 other than the ones described above.
The number and lengths of rods and arms in one or more of the embodiments can be varied.
The relatively stiff components described above could be made of a resorbable material so that their stiffness would change over time.
The rods 30 and 32 could be flexible or rigid.
In the embodiment of FIG. 9, the adjustment mechanism for moving the assembly consisting of the bracket 64 and the arms 66a and 66b axially may be on the latter assembly rather than the body member 62.
In the embodiment of FIG. 10, the arms 74a and 74b could be pivotally mounted to the body member 52.
The assemblies of the above embodiments can be implanted between body portions other than vertebrae.
The assemblies of the above embodiments can be inserted between two vertebrae following a discectomy in which a disc between the adjacent vertebrae is removed, or corpectomy in which at least one vertebrae is removed.
The spatial references made above, such as “under”, “over”, “between”, “flexible, soft”, “lower”, “top”, “bottom”, etc. are for the purpose of illustration only and do not limit the specific orientation or location of the structure described above.

The preceding specific embodiments are illustrative of the practice of the invention. It is to be understood, therefore, that other expedients known to those skilled in the art or disclosed herein, may be employed without departing from the invention or the scope of the appended claims, as detailed above. In the claims, means-plus-function clauses are intended to cover the structures described herein as performing the recited function and not only structural equivalents but also equivalent structures. Thus, although a nail and a screw may not be structural equivalents in that a nail employs a cylindrical surface to secure wooden parts together, whereas a screw employs a helical surface, in the environment of fastening wooden parts a nail and a screw are equivalent structures.

What is claimed is:

1. A prosthetic assembly for insertion in a spinal column, the device comprising:
at least one rod secured to the spinal column;
a spacer engaging a spinous process of a vertebrae of the spinal column; and
an adapter connected to the rod and engaging the spacer.

2. The assembly of claim 1 wherein the spacer is supported between the spinous process and the adapter.

3. The assembly of claim 1 wherein the adapter comprises a body member engaging the spacer, and at least one arm extending from the body member and engaging the rod.

4. The assembly of claim 3 wherein there are two rods and two arms that respectively engage the rods.

5. The assembly of claim 3 wherein the position of the arm relative to the body member and the rod is adjustable.

6. The assembly of claim 5 wherein the distance of the arm from the body member to the rod is adjustable.

7. The assembly of claim 5 wherein the rod is rotatable relative to the body member and the rod.

8. The assembly of claim 1 wherein the spinal column includes an additional vertebrae adjacent the first-mentioned vertebrae, wherein the additional vertebrae does not have a spinous process, and wherein the spacer and the adapter stabilize the spinal column between the two vertebrae.

9. The assembly of claim 8 wherein the rod is connected to the two vertebrae.

10. The assembly of claim 9 wherein there are two rods each of which is connected to the two vertebrae, and further comprising two arms on the adapter that respectively engage the two rods.

11. The assembly of claim 8 further comprising a retainer disposed at the respectively ends of the rod and a screw extending through the retainer and into the vertebrae to connect the rod to the vertebrae.

12. The assembly of claim 11 further comprising at least one arm extending from the adapter and connected to the retainer.

13. The assembly of claim 12 wherein a opening is formed through one end portion of the arm for receiving the screw, to connect the arm to the retainer.

14. The assembly of claim 1 wherein two notches are provided on the spacer for receiving the spinous process and the adapter, respectively.

15. The assembly of claim 1 wherein the position of the adapter relative to the spinous process, the spacer, and the rod is adjustable to insure a good fit between the spacer and adapter.

16. A method for implanting a prosthetic assembly in a spinal column, the method comprising:
securing at least one rod to the spinal column;
engaging a spinous process of a vertebrae of the spinal column with a spacer; and
connecting the rod to the spacer.

17. The method of claim 16 wherein the rod is connected to the spacer by an adapter.

18. The method of claim 17 further comprising supporting the spacer between the spinous process and the adapter.

19. The method of claim 18 further comprising providing at least one arm on the adapter that engages the rod.

20. The method of claim 19 wherein there are two rods and two arms that respectively engage the rods.

21. The method assembly of claim 19 further comprising adjusting the position of the arm relative to the body member and the rod.

22. The method of claim 19 further comprising adjusting the distance of the arm from the body member to the rod.

23. The method of claim 19 rotating the arm relative to the body member and the rod to adjust the angular position of the arm.

24. The method of claim 19 further comprising providing a retainer at the respectively ends of the rod, and driving a screw through the retainer and into the vertebrae to connect the rod to the vertebrae.

25. The assembly of claim 24 further comprising extending the arm from the adapter and connecting the arm to the retainer.

26. The assembly of claim 25 further comprising extending the screw through an opening formed through one end portion of the arm to connect the arm to the retainer.

27. The method of claim 16 wherein the adapter includes a body member and a bracket connected to the body member, and further comprising adjusting the spacing between the bracket and the body member.

28. The method of claim 16 wherein the spinal column includes an additional vertebrae adjacent the first-mentioned vertebrae, wherein the additional vertebrae does not have a spinous process, and wherein the spacer and the adapter stabilize the spinal column between the two vertebrae.

29. The method of claim 28 wherein the rod is connected to the two vertebrae.

30. The method of claim 28 wherein there are two parallel rods each connected to the two vertebrae, and further comprising providing two arms on the adapter that respectively engage the two rods.

31. The method of claim 16 further comprising providing two notches on the spacer for receiving the spinous process and the adapter, respectively.

32. The method of claim 16 further comprising adjusting the position of the adapter relative to the spinous process, the spacer, and the rod to insure an optimum fit between the spacer and adapter.

33. A method for percutaneously or subcutaneously implanting a prosthetic assembly in a spinal column, the method comprising:
securing at least one rod to the spinal column;
engaging a spinous process of a vertebrae of the spinal column with a spacer; and
connecting the rod to the spacer.
